(19) United States
(12) Patent Application Publication
(10) Pub. No.: US 2019/0336516 A1
(43) Pub. Date: Nov. 7, 2019

(54) MODULATION OF TUMOR CELL SUSCEPTIBILITY

(71) Applicant: NANT HOLDINGSP IP, LLC, Culver City, CA (US)

(72) Inventor: Patrick SOON-SHIONG, Culver City, CA (US)

(21) Appl. No.: 16/478,628

(22) PCT Filed: Jan. 18, 2018
(86) PCT No.: PCT/US2018/014277

§ 371 Date: Jul. 17, 2019

Related U.S. Application Data
(60) Provisional application No. 62/447,818, filed on Jan. 18, 2017.

(51) Int. Cl. A61K 31/704; A61K 45/06; A61K 31/713; A61K 39/395; A61K 35/17; A61K 39/235; C07K 16/24

(52) U.S. Cl. CPC A61K 31/704; A61K 45/06; A61K 31/713; C07K 2317/622; A61K 35/17; A61K 39/235; C07K 16/244; A61K 39/3955

(57) ABSTRACT

Contemplated compositions and methods sensitize tumor cells to a cancer treatment regimen, including chemotherapy, radiation therapy, and immune therapy by preventing EMT (epidermal to mesenchymal transition) of the tumor cell, or by reversing the tumor cell from a mesenchymal to an epidermal state. Thusly sensitized cells are then subjected to the cancer treatment regimen.

This application claims priority to copending U.S. provisional application with the Ser. No. 62/447,818, filed Jan. 18, 2016, and which is incorporated by reference herein.

### FIELD OF THE INVENTION

The field of the invention is treatment of a tumor, and especially as it relates to treatments and methods that precondition tumor cells to be more sensitive to chemotherapy, radiation, and/or immune therapy.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Cancer stem cells are a subgroup of cells within a tumor and have ability to self-renew and differentiate to any types of cells in a particular type of tumor to so initiate and sustain the formation and growth of cancer. In many instances, cancer stem cells will cause relapse and metastasis of the tumor, which often also acquires treatment resistance during such process. Several hypotheses have been proposed for the generation of cancer stem cells. Among those, the de-differentiation hypothesis suggests that a mutated cell can be de-differentiated to obtain stem cell-like characteristics. For example, a tumor cell can be transformed to a precursor cell for metastatic cancer cell or cancer stem cell via epithelial-mesenchymal transition (EMT).

EMT is a physiological process during embryogenesis that appears to be reinstated in adult tissues undergoing wound healing and tissue regeneration, or under certain pathological conditions such as fibrosis and cancer. Tumor EMT involves a phenotypic switch that promotes acquisition of a fibroblastoid-like morphology by epithelial tumor cells, that reduces cell polarity and cell-to-cell contacts, and that decreases expression of epithelial markers, including E-cadherin and cytokeratins. On the other hand, epithelial tumor cells undergoing EMT will typically gain expression of mesenchymal-associated proteins, such as fibronectin and vimentin, and will have enhanced cell motility, invasiveness, and metastatic propensity in vivo. Tumor EMT has also been shown to contribute to the acquisition of tumor resistance to chemotherapy, radiation, and certain small-molecule-targeted therapies, thus representing a major mechanism contributing to the progression of carcinomas.

Various signaling pathways in the tumor cell are known or suspected to be related to the induction and/or maintenance of tumor EMT. For example, the IL-8/IL-8 receptor axis was investigated with respect to the induction and/or maintenance of tumor EMT and its ability to remodel the tumor microenvironment. For example, autocrine loops of IL-8 were suggested to induce and maintain tumor EMT (see e.g., Future Oncol 2012, 8(6): 713-722). Therefore, pharmaceutical intervention targeting IL-8 signaling was suggested as a therapeutic approach to halt disease progression driven by IL-8 and other CXCR1/2 ligands (see e.g., Breast Cancer Research 2013, 15:210). Similarly, the IL-8/CXCR1 axis was reported to be associated with cancer stem cell-like properties and to correlate with the clinical prognosis in human pancreatic cancer cases (see e.g., Scientific Reports 2014, 4: 5911), and it was suggested to target pancreatic cancer stem cells by disrupting the IL-8/CXCR1 axis. Interestingly, IL-8 is also a potent chemoattractant for neutrophils and monocytes and has been implicated in directing myeloid derived suppressor cells into the tumor microenvironment (see e.g., Clin Cancer Res 2016, and Vaccines 2016, 4, 22). In yet another example, some myeloid-derived suppressor cells (MDSCs) preferentially infiltrate the tumor and actively induce EMT via transforming growth factor (TGF)-β, epithelial growth factor (EGF) and/or hepatocyte growth factor (HGF)-mediated pathways. However, IL-8 signaling inhibition alone or MDSC inhibition alone has not led to a therapeutically effective path in the treatment of cancer.

Thus, even though the role of EMT in acquiring stem-ness of tumor cell and resistance to different types of cancer treatment has been extensively studied, none of the insights have led to a therapeutically effective treatment that would help eradicate the tumor, let alone a treatment regimen to inhibit or reverse EMT so that the treatment of tumor cells can be more effective. Therefore, there is still a need for compositions and methods that improve therapy outcome for treatment of a tumor.

### SUMMARY OF THE INVENTION

The inventive subject matter is directed to various compositions and methods in which tumor cells are preconditioned to increase the effectiveness of cancer treatment(s) including chemotherapy, radiation therapy, and/or immune therapy of the tumor cells. More particularly, the inventors have discovered that the tumor cell's resistance to such cancer treatment(s) can be substantially reduced by inhibiting or even reversing EMT of the tumor cells. The inventors further discovered that EMT of the tumor cells can be effectively inhibited or even reversed by treating the tumor cells or tumor with an agent that blocks myeloid derived suppressor cells and one or more agents that blocks IL-8 signaling, CXCR1 pathway activity, or CXCR2 pathway activity. Thusly treated tumor cells are expected to exhibit reduced stemness and are therefore expected to be significantly more sensitive to chemotherapy, radiation therapy, and/or immune therapy.

Thus, one aspect of the inventive subject matter includes a method of preconditioning a tumor microenvironment prior to a treatment of a tumor cell. In this method, the tumor cell is contacted with a first reagent that suppresses a myeloid derived suppressor cell in a tumor microenvironment, and contacted with a second reagent that blocks at least one of IL-8 signaling, a CXCR1 signaling pathway, and a signaling CXCR2 pathway. The first and second agents are administered in first and second amounts that prevent epidermal to mesenchymal transition of the tumor cell. In some embodiments, the first reagent is administered to the tumor prior to the second reagent. In other embodiments, the second reagent is administered to the tumor prior to the first reagent. In still other contemplated embodiments, the first and second reagents can be administered simultaneously or substantially simultaneously such that the tumor is treated with the first and second reagents simultaneously or substantially simultaneously.

In further contemplated aspects, the first reagent can be a myeloid derived suppressor cell recruitment inhibitor, a myeloid derived suppressor cell expansion inhibitor, a myeloid derived suppressor cell differentiation inhibitor, or a myeloid derived suppressor cell activity inhibitor. The second reagent can be an IL-8 antagonist, a CXCR1 inhibitor, and/or a CXCR2 inhibitor. It is contemplated that the IL-8 antagonist, the CXCR1 inhibitor, and/or the CXCR2 inhibitor can be an antibody or a small nucleotide inhibiting the activity of IL-8-mediated or CXCR1/CXCR2-mediated signaling pathways. It is generally preferred that the treatment includes chemotherapy, radiation therapy, and/or immune therapy (e.g., inducing NK cell-mediated immune response and a T cell-mediated immune response, etc.). In some embodiments, the inventors contemplate that the first and/or second reagent can be coupled with a molecule binding to the tumor cell to so specifically target tumor cell or tumor microenvironment when systemically administered.

In another aspect of the inventive subject matter, the inventor also contemplates a method of treating a tumor cell in a patient. Preferred methods will comprise a step of preconditioning the tumor cell by contacting the tumor cell with a first reagent that suppresses a myeloid derived suppressor cell in a tumor microenvironment, and contacting the tumor cell with a second reagent that blocks at least one of IL-8 signaling, a CXCR1 pathway, a CXCR2 pathway. The first and second agents are administered in first and second amounts that prevent epidermal to mesenchymal transition of the tumor cell. Once the tumor cell or tumor is preconditioned, then the tumor cell can be treated with at least one of chemotherapy, radiation therapy, and an immune therapy.

Various objects, features, aspects and advantages of the inventive subject matter will become more apparent from the following detailed description of preferred embodiments.

### DETAILED DESCRIPTION

The inventors now discovered that tumor cells and/or a tumor microenvironment can be preconditioned to increase the effectiveness of cancer treatment against the tumor by preventing or even reversing the EMT of the tumor cells. Viewed from a different perspective, where the stemness of a tumor or tumor stem cell is reduced, or where the tumor microenvironment is more resistant to induction of EMT of a tumor cell, treatment of the tumor or tumor stem cells with one or more of chemotherapy, radiation therapy, and immune therapy is more effective.

As used herein, the term “tumor” refers to, and is interchangeably used with one or more cancer cells, cancer tissues, malignant tumor cells, or malignant tumor tissue, that can be placed or found in one or more anatomical locations in a human body. As used herein, the term “bind” refers to, and can be interchangeably used with a term “recognize” and/or “detect”, an interaction between two molecules with a high affinity with a K_{D} of equal or less than 10⁻⁶M, or equal or less than 10⁻⁷M. As used herein, the term “provide” or “providing” refers to and includes any acts of manufacturing, generating, placing, enabling to use, or making ready to use.

In an especially preferred aspect of the inventive subject matter, the inventors contemplate preconditioning of tumor microenvironment prior to a tumor cell treatment to induce sensitization of the tumor cell and/or tumor microenvironment to the treatment. Preferably, the tumor or tumor cell is treated with one formulation or a reagent that inhibits myeloid derived suppressor cells (MDSCs) and with another formulation or a reagent that blocks IL-8-mediated signaling, CXCR1 pathway activity or CXCR2 pathway activity. Most typically, it is contemplated that the tumor is sequentially contacted with the formulation or a reagent that inhibits MDSCs, and then contacted with another formulation or a reagent that blocks IL-8-mediated signaling, CXCR1 pathway activity or CXCR2 pathway activity.

For example, the tumor can be treated with the formulation or reagent that inhibits the MDSCs at least 6 hours, at least 12 hours, at least 24 hours, at least 36 hours, at least 3 days, at least 7 days before being treated with the formulation or reagent that blocks IL-8-mediated signaling, CXCR1 pathway activity or CXCR2 pathway activity. Conversely, in further aspects of the inventive subject matter, it is also contemplated that the tumor is treated with the formulation or a reagent that blocks IL-8-mediated signaling, CXCR1 pathway activity or CXCR2 pathway activity, and then contacted with the formulation or a reagent that inhibits MDSCs. For example, the tumor can be contacted with contacted with the formulation or a reagent that blocks IL-8-mediated signaling, CXCR1 pathway activity or CXCR2 pathway activity at least 6 hours, at least 12 hours, at least 24 hours, at least 36 hours, at least 3 days, at least 7 days before being contacted with the formulation or a reagent that inhibits MDSCs. In still other embodiments, the tumor may be contacted with the formulation or a reagent that blocks MDSCs and another formulation or a reagent that blocks IL-8-mediated signaling, CXCR1 pathway activity or CXCR2 pathway activity simultaneously or substantially simultaneously (e.g., within 3 hours, within 1 hour, within 30 min, within 10 min, etc.).

With respect to compounds and compositions suitable for use herein, it should be noted that any reagents and/or formulations that inhibit MDSCs, IL-8-mediated signaling, CXCR1 pathway activity, and/or CXCR2 pathway activity are contemplated herein. For example, with respect to contemplated inhibitors of MDSCs, it is preferred that the reagent(s) can preferentially, or even selectively, inhibit granulocytic MDSC. Yet, it is also contemplated that any reagents that can effectively inhibit any other types of MDSCs in the tumor and/or tumor microenvironment can be used. In addition, it should be appreciated that suitable reagent(s) can act in various manners, including inhibiting recruitment of MDSCs to the tumor, inhibiting expansion of MDSC in the tumor, inhibiting differentiation of MDSCs, and/or inhibiting activity (e.g., secreting chemokines, etc.) at the tumor, or even eliminating or reducing the number of MDSCs in the tumor or tumor microenvironment. For example, MDSC inhibitors may reduce or abrogate recruitment of MDSCs to the tumor and/or accumulation of MDSCs in the tumor, which may be achieved by administration of one or more antagonists of one or more colony-stimulating factor 1 receptor (CSF-R), granulocyte colony-stimulating factor (G-CSF), C-C motif chemokine ligand 2 (CCL2), or C-X-C chemokine receptor type 4 (CXCR4). As used herein, antagonist refers any molecule that is capable to directly or indirectly inhibit the activity of target molecule. Thus, antagonist may include small molecule inhibitors, antibodies or fragments thereof that bind to the target molecule, single-chain variable fragment (scFv) molecule binding to the target molecule, or any other suitable binding molecules. For example, the antagonist of CSF-R may include a small molecule inhibitor (e.g., Pexidartinib, etc.) or one or more monoclonal antibodies against CSF-R (e.g., Emactuzumab, AMG820, imc-CS4, MCS110, etc.).

Alternatively or additionally, expansion of the MDSCs in the tumor may be inhibited by administering gemcitabine, amino bisphosphonates, sunitinib, or celecoxib, and differentiation of MDSCs in the tumor may be inhibited by taxanes, curcumin, or Vitamin D3. In addition, MDSC activity in the tumor may be inhibited by administration of amiloride, CpG, COX2 inhibitors, PDE-5 inhibitors, or PGE2 inhibitors. Further, MDSCs in the tumor or tumor microenvironment can be eliminated or at least reduced by treating the tumor with doxorubicin (or aldoxorubicin for enhanced activity in the acidic tumor microenvironment). As used herein, the term “administering” the formulation refers to both direct and indirect administration of the formulation, wherein direct administration of the formulation is typically performed by a health care professional (e.g., physician, nurse, etc.), and wherein indirect administration includes a step of providing or making available the formulation to the health care professional for direct administration (e.g., via injection, etc.).

In some embodiments, it is contemplated that different types of MDSC inhibitors can be administered together to act on the MDSCs in different manner, preferably at different time points. For example, inhibitors for recruitment/accumulation of MDSCs and inhibitors of MDSC activity can be coupled with different types of carrier (e.g., albumin-linked, encapsulated in a lipid micelle, cell-penetrating peptide-linked, etc.) such that the inhibitors for recruitment and/or accumulation of MDSCs acts on the tumor prior to the inhibitors of MDSC activity by differential access rate to the tumor or different diffusion rate of the reagents. In this scenario, it is contemplated that the recruitment of the MDSCs to the tumor is blocked first and then the activity of pre-existing MDSCs in the tumor is blocked such that the effect of MDSC activity in the tumor can be effectively eradicated. In other embodiments, different types of MDSC inhibitors can be administered by different methods of administration to act on the MDSCs at different time points. For example, the inhibitors for recruitment/accumulation of MDSCs can be injected intratumorally (e.g., especially where the tumor is a solid tumor, and the tumor cell is from the solid tumor) and the inhibitors of MDSC activity can be injected intravenously (or any other systemic injection).

With respect to the reagent(s) that blocks IL-8-mediated signaling, CXCR1 pathway activity or CXCR2 pathway activity, it should be appreciated that suitable agents can act in various manners, including trapping tumor cell secreted IL-8, reducing the expression of IL-8 from the tumor cell, blocking the IL-8 binding to the CXCR1/2, inhibiting signaling cascade mediated by CXCR1/2. For example, tumor cell secreted IL-8 or cell-free IL-8 from other sources can be captured by any monoclonal or polyclonal IL-8 antibodies (see e.g., J. Immunol. Methods 1992 149:227 or WO 1997/001354), a scFv molecule (scFv fragment itself or as a conjugate or hybrid molecule with a superagonist molecule (e.g., ALT-803, TxM, from Altor bioscience, Inc., etc.)) binding to IL-8, or any other non-antibody binding molecules of IL-8 that can be identified by RNA display. In still another example, tumor cell secreted IL-8 can be decreased by reducing the cellular expression of IL-8 by introducing one or more regulatory RNA molecule (e.g., via RNA interference using shRNA, siRNA, or miRNA) into the IL-8 expressing cells (e.g., tumor cells, etc.).

Alternatively or additionally, IL-8-mediated signaling cascade through CXCR1/2 can be inhibited by blocking the binding of IL-8 to the IL-8 receptor including CXCR1/2 or inhibiting CXCR1/2 activation. For example, IL-8 binding to the CXCR1/2 can be inhibited by IL-8 receptor antagonists (e.g., CXCR1 antagonist, CXCR2 antagonist, etc.) including various 2-amino-3-heteroaryl-quinoxalines (see e.g., Bioorg Med Chem. 2003 Aug. 15; 11(17):3777-90), 6-Chloro-3-[[[(2,3-dichlorophenyl)amino]carbonyl]amino]-2-hydroxybenzenesulfonamide (SB332235), or N-(2-Bromophenyl)-N′-(7-cyano-1H-benzotriazol-4-yl)urea (SB265610). If inhibitors with higher specificity are desired, SCH-527123 and SCH-479833 may be employed that will selectively inhibit CXCR2 and CXCR1, respectively (see e.g., Clin Cancer Res. 2009 Apr. 1; 15(7):2380-6). In still another example, the activation of the IL-8 receptor, including CXCR1/2, can be inhibited using reparixin (also known as repertaxin, see e.g., Biol Pharm Bull. 2011; 34(1):120-7), or the IL-8-mediated signaling cascade through CXCR1/2 can be inhibited by blocking one or more elements in the signaling pathways. Thus, inhibitors can also target CXCR1 and 2 signaling pathways by inhibiting or interfering with PI3-K, pAkt, or mTOR for CXCR1 signaling, and/or by inhibiting or interfering with RhoGTPase, RacGTPas, and Ras, Raf, Mek, or pErk for CXCR2 signaling.

In some embodiments, it is contemplated that different types of IL-8, CXCR1, CXCR2 inhibitors can be administered together to act on the IL-8 mediated signaling pathway to boost the therapeutic effect. For example, IL-8 binding scFv fragment or antibodies can be formulated together, or at least administered together with one or more CXCR1 or CXCR2 antagonist such that EMT-enhancing signaling pathway via IL-8, CXCR1, or CXCR2 can be inhibited by both loss of ligands (IL-8) and loss of receptor function (e.g., via binding of ligand other than IL-8).

Of course, where the different types of IL-8, CXCR1, CXCR2 inhibitors are administered to inhibit IL-8 mediated signaling pathway, the timing and sequence of administering of different types of inhibitors may vary. For example, where the IL-8 inhibitor is recombinant nucleic acid encoding siRNA against IL-8 transcript and the CXCR1/CXCR2 inhibitor is reparixin, the recombinant nucleic acid would be effectively delivered by transfecting the IL-8 secreting cells (e.g., tumor cell) by generating the recombinant virus (e.g., adenoviruses, lentiviruses, adeno-associated viruses, parvoviruses, togaviruses, poxviruses, herpes viruses) containing the recombinant nucleic acid, rather than naked siRNA in a liquid carrier (e.g., saline solution, etc.). Thus, in such embodiment, the IL-8 inhibitor (siRNA) can be administered as recombinant virus either intravenously or intratumorally at least 1 day, at least 3 days, at least 7 days prior to administering the reparixin intravenously.

Additionally, where the specific targeting of any reagents to the tumor cell or tumor microenvironment is desired, it is contemplated that the reagent can be directly conjugated or indirectly coupled with a binding molecule (e.g., an antibody, a scFv molecule, etc.) to a tumor associated antigen expressed on the tumor cell surface. Preferably, the tumor associated antigen is a patient-specific, tumor-specific neoepitope that is identified by analyzing omics data obtained from the tumor sample of the patient. For example, a scFv molecule binding to a tumor neoepitope can be generated by first identifying the nucleic acid sequence of V_{H} and V_{L} specific to the tumor neoepitope. In some embodiments, a nucleic acid sequence of V_{H} and V_{L} can be identified from a monoclonal antibody sequence database with known specificity and binding affinity to the tumor epitope. Alternatively, the nucleic acid sequence of V_{H} and V_{L} can be identified via an in silico analysis of candidate sequences (e.g., via IgBLAST sequence analysis tool, etc.). In other embodiments, the nucleic acid sequence of V_{H} and V_{L} can be identified via a mass screening of peptides having various affinities to the tumor neoepitope, tumor associated antigen, or self-lipid via any suitable in vitro assays (e.g., flow cytometry, SPR assay, a kinetic exclusion assay, etc.).

In an embodiment where the scFv molecule binding to a tumor neoepitope is coupled with one or more reagent(s) (e.g., MDSC inhibitor, IL-8 inhibitor, or CXCR1/2 inhibitor, etc.), the inventors contemplate a nanoparticle can be used as an intermediate molecule to couple the scFv and the reagent. For example, suitable nanoparticles may include non-protein beads (e.g., a gold nanoparticle, etc.) and protein beads (e.g., protein A, protein G, protein Z, albumin, refolded albumin). Especially, where the carrier protein is an albumin, a hydrophobic reagent may fit in one of Sudlow site I and II of the albumin or any other hydrophobic area of the albumin. In some embodiments where the reagent is not hydrophobic enough, it is contemplated that the reagent can be coupled with an hydrophobic short anchor peptide (e.g., having a length of at least 10 amino acids, 15 amino acids, 20 amino acids, 30 amino acids, etc.) such that the reagent can bind the Sudlow site I or II of the albumin via the hydrophobic short anchor peptide.

It is contemplated that some reagents that inhibits MDSCs or that inhibits IL-8 or CXCR1/2 may cross-react or affect more than one target (e.g., two element in the signaling pathways, two signaling pathways, etc.). For example, eliminating or substantially reducing IL-8 availability by trapping IL-8 inhibits IL-8 itself, and also may inhibit CXCR1 and/or CXCR2 by depleting the ligands. In addition, eliminating or substantially reducing IL-8 availability may affect the recruitment of MDSCs to the tumor.

Additionally, the inventors further contemplate administering another reagent that inhibit EMT of the tumor cell or reverse the EMT process of the tumor cell, or even promote mesenchymal to epithelial transition (MET) of the tumor cell. For example, during the EMT process, TGF-β induces isoform switching of FGF Receptor 2 (e.g., from isotype IIIb to IIIc), and it is contemplated that inhibiting TGF-β activity in the tumor cells (e.g., using dominant negative form of TGF-β RII, monoclonal antibodies against TGF-beta 1 and beta 2, including lerdelimumab and metelimumab, etc.) may reduce or prohibit the isoform switching of FGF Receptor 2 to so prevent EMT of the tumor cell. In another example, MET may be induced in vitro by administering 8-bromo-cAMP, Taxol, or Adenosine 3prime,5prime-cyclic Monophosphate, N6-Benzoyl-Sodium Salt, which activate protein kinase A (PKA). MET of the tumor cell can be also induced by administering a recombinant virus encoding recombinant E-Cadherin or regulatory RNA inhibiting N-Cadherin expression to stimulate of E-Cadherin overexpression and reduce N-Cadherin expression. Further, MET of the tumor cell can be also induced by EGFR inhibition and/or down-regulation of Snail, Slug, Zeb-1, Zeb-2, and/or N-cadherin (e.g., using siRNA, miRNA, shRNA, or other regulatory small molecule reducing the post-transcriptional expression, etc.).

It is contemplated that such additional reagent can be contacted with the tumor in any suitable time. For example, the additional reagent that inhibit EMT, reverse EMT, or promote MET can be contacted with the tumor after the MDSC inhibitor is contacted with the tumor, after the IL-8, CXCR1/2 inhibitor is contacted with the tumor, or both MDSC inhibitor and IL-8 and/or CXCR1/2 inhibitor are contacted with the tumor. Therefore, the additional reagent that inhibit EMT, reverse EMT, or promote MET can be contacted with the tumor instead of one of MDSC inhibitor and IL-8 and/or CXCR1/2 inhibitor.

It should be appreciated that the order and route of administrating one or more reagents to the patient may vary considerably. For example, reagent A can be administered by intravenous injection and reagent B can be administered intratumoral injection. In such scenario, even if reagent A may be administered prior to the reagent B, reagent B may act on the tumor earlier or more effectively than reagent A. In another example, reagent C and reagent D may be injected intratumorally at the same time, but reagent C may contact the tumor prior to reagent D where the reagent D is packaged in a carrier that allows for slow diffusion or release of the reagent.

As will be readily appreciated, with respect to dose and schedule of the formulation administration, it is contemplated that the dose and/or schedule may vary depending on the type of reagents, packaging of the reagents, administration method of the reagents, type and prognosis of disease (e.g., tumor type, size, location), and health status of the patient (e.g., including age, gender, etc.). Most preferably, the agents contemplated herein will be administered in an amount and at a schedule such that epidermal to mesenchymal transition of the tumor cells in the tumor is reduced or prevented (at least 10%, at least 20%, at least 30%, at least 50%, at least 70%, compared to non-preconditioned tumor, etc.), and/or in an amount and at a schedule such the tumor cell in the tumor is driven from a mesenchymal to an epidermal state (e.g., at least 10%, at least 20%, at least 30%, at least 50%, at least 70% of tumor cell undergone EMT are reversed via MET, etc.). It is also preferred that the dose and schedule may be selected and regulated so that the formulation does not provide any significant toxic effect to the host normal cells, yet sufficient to be elicit sensitization of the tumor cells to the chemotherapy, radiation therapy, or an immune therapy.

There are numerous manners to ascertain the state of a tumor cell, and all of such known methods are deemed suitable for use herein (which may also be used to determine appropriate dosages and schedules). For example, and viewed from a morphological perspective, EMT is the process whereby epithelial cells lose their characteristic epithelial features (such as apical-basolateral polarity, extensive intercellular adhesions, and contact growth inhibition) in favor of acquiring mesenchymal features (such as leading edge-trailing edge asymmetry, loose intercellular contacts, and motility/invasiveness). On a cellular level, EMT is often accompanied with overexpression of E-Cadherin relative to N-Cadherin on the cell surface. On a molecular biological level, EMT is often accompanied by EMT transcription factors, including zinc-finger proteins such as Snail, Slug, ZEB1, and ZEB2, and helix-loop-helix transcription factors Twist1 and Twist2.

Thus, in some embodiments, the dose and schedule of the formulation administration may be determined or adjusted by determining morphological and/or molecular biological changes of the tumor cells between contacting with MDSC inhibitor and contacting with IL-8, CXCR1/2 inhibitors. In some embodiments, where the tumor is contacted with the MDSC inhibitor first and then contacted with IL-8, CXCR1/2 inhibitors, a biopsy sample of the tumor can be obtained after the initial MDSC inhibitor treatment to the tumor. For example, the biopsy tissue can be further processed for either immunohistochemical assays or biochemical assays (e.g., fix and slice the biopsy tissue, etc.) and the expression level and/or distribution of EMT marker, for example, ratio and distribution of E-Cadherin and/or N-Cadherin, can be quantitatively and/or qualitatively assessed. Preferably, the expression level and/or distribution of E-Cadherin and/or N-Cadherin can be compared with those of biopsy tissue before MDSC inhibitor treatment. Based on any change of E-Cadherin and/or N-Cadherin levels and/or distribution, the treatment regimen of IL-8, CXCR1/2 inhibitors or extended MDSC inhibitor may be determined. For other example, the biopsy tissue can be further processed for immunohistochemical assays and the quantity and distribution of MDSCs in the tumor microenvironment can be analyzed using MDSC markers (e.g., Siglec-3/CD33, etc.) to determine whether accumulation of MDSC could be effectively prohibited by MDSC inhibitors

Without wishing to be bound by any specific theory, the inventors contemplate that pre-conditioning of tumor or tumor microenvironment with an MDSC inhibitor and IL-8, CXCR1/2 inhibitors (or MET promoting reagent) can prevent EMT of the tumor cells or even reverse the tumor cells that had been transformed via EMT process. For example, some MDSCs preferentially infiltrate the tumor and actively induce EMT via transforming growth factor (TGF)-β, epithelial growth factor (EGF) and/or hepatocyte growth factor (HGF)-mediated pathways. Thus, preconditioning of tumors with MDSC inhibitor(s) to inhibit MDSC activity in the tumor microenvironment first may at least slow down the EMT of tumor cells via EGF/TGF-β or HGF-mediated pathways. Then contacting IL-8, CXCR1/2 inhibitors may further prevent EMT of tumor cells as well as recruitment or accumulation of MDSCs in the tumor environment, as IL-8 play a key role as a chemoattractant of MDSC to the tumor. Conversely, contacting IL-8, CXCR1/2 inhibitors first may effectively prevent EMT via CXCR1/2-mediated pathways in the tumor microenvironment, and can further prevent recruitment or accumulation of MDSCs in the tumor environment. Then, contacting with MDSC inhibitor(s) may further slow down the EMT of tumor cells via EGF/TGF-β or HGF-mediated pathways by existing MDSCs in the tumor microenvironment.

It is contemplated that preventing EMT or reversing EMT of tumor cells may effectively prevent the tumor cells to acquire the resistance or to reduce sensitivity to various cancer therapies including chemotherapy, radiation therapy or immune therapy. In other words, by preventing EMT or reversing EMT of tumor cells, tumor cells in the tumor may be sensitized to such various cancer therapies. For example, cancer therapies to the preconditioned tumor may have increased the therapy effectiveness at least 10%, at least 20%, at least 30%, at least 50%, at least 70% compared to the therapies treated to a tumor that are not preconditioned, when the therapy effectiveness is determined by reduced tumor size, reduced metastasis rate, reduced growing rate, reduced number of circulating tumor cells, etc.). As used herein, chemotherapy includes any type of chemotherapy, preferably targeted chemotherapy, and/or low-dose metronomic chemotherapy as best suitable for the particular tumor. Radiation therapy includes an external beam radiation therapy and an internal radiation therapy (e.g., brachytherapy, systemic therapy, etc.).

Consequently, it should be appreciated that the compositions and methods to precondition a tumor or tumor cell are not intended as a treatment of a cancer, or even intended to be used as a treatment of the cancer. Instead, the compositions and methods presented herein are intended to precede one or more cancer treatments, and to render the tumor cells or the tumor more sensitive to subsequent cancer treatment. Viewed from a different perspective, the administration of the compounds and compositions herein will increase the therapeutic effect of a subsequent cancer treatment (as compared to not preconditioned tumors or tumor cells).

Immune therapy, as used herein, includes any types of immune therapy that may elicit an NK-cell mediated immune response, an NKT-cell mediated immune response, and/or a T-cell mediated immune response. Therefore, the immune therapy may include administering a cancer vaccine (e.g., viral vaccine, bacterial vaccine, yeast vaccine, etc.), administering one or more immune-stimulatory molecules (e.g., CD80, CD86, CD30, CD40, CD30L, CD40L, ICOS-L, B7-H3, B7-H4, CD70, OX40L, 4-1BBL, GITR-L, TIM-3, TIM-4, CD48, CD58, TL1A, ICAM-1, and LFA3, etc.), immune stimulatory cytokines (e.g., IL-2, IL-12, IL-15, IL-15 super agonist (ALT803), IL-21, IPS1, and LMP, etc.), and/or checkpoint inhibitors (e.g., antibodies or binding molecules to CTLA-4 (especially for CD8⁺ cells), PD-1 (especially for CD4⁺ cells), TIM1 receptor, 2B4, and CD160, etc.). In addition, contemplated immune therapies include any cell-based therapies such as administration of NK cells, genetically engineered NK cells, and especially aNK cells, haNK cells, optionally with bound antibody, or taNK cells, NKT cells, genetically engineered NKT cells, (re-)activated T cells or T cells expressing a chimeric antigen receptor, and/or dendritic cells expressing cancer neoepitopes or cancer specific or associated antigens.

Therefore, the inventors also contemplate that a patient can be treated with at least one or more cancer therapies including chemotherapy, a radiation therapy, or immune therapy after preconditioning the tumor or tumor environment with MSDC inhibitors, IL-8 inhibitors and/or CXCR1/2 inhibitors (or MET promoters). The treatment regimen and schedule may vary depending on the type(s) of preconditioning and cancer therapies. For example, a chemotherapy or a radiation therapy may be administered to a patient at least 1 day, 3 days, 5 days, 7 days after completing preconditioning of the tumor. In another example, in some embodiments, a cell-based immune therapy may be administered to a patient at least 1 day, 3 days, 5 days, 7 days after completing preconditioning of the tumor. In still other embodiments, depending on the type of cell-based immune therapy, the cell-based immune therapy may be administered to the patient by completion of preconditioning of the tumor or even during the preconditioning of the tumor (e.g., at least 12 hours, at least 1 day, at least 3 days after beginning of preconditioning, etc.).

Most typically, treatment effect (e.g., as measured by tumor mass or volume, number of metastases, number of circulating tumor cells) will be at least 10%, more typically at least 20%, and even more typically at least 30% improved as the same treatment without use of the compositions and methods presented herein. In addition, it should be appreciated that the compositions and methods presented herein may also significantly reduce or even eliminate tumor growth or dissemination due to the reduction of tumor stem cells.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1.-40. (canceled)

41. A method of preconditioning a tumor microenvironment prior to a treatment to a tumor cell, comprising:
contacting the tumor cell with a first reagent that suppresses a myeloid derived suppressor cell (MDSC) in a tumor microenvironment;
contacting the tumor cell with a second reagent that blocks at least one of IL-8-mediated signaling pathway, a CXCR1 signaling pathway, a CXCR2 signaling pathway;
wherein the first and second agents are administered in a first and second amounts that prevent epidermal to mesenchymal transition of the tumor cell;
wherein the first reagent reduces or abrogates recruitment of MDSCs in the tumor and/or accumulation of MDSCs in the tumor, and/or inhibits expansion of MDSCs in the tumor, and
wherein the first reagent is distinct from the second reagent.

42. The method of claim 41, wherein the first reagent is contacted with the tumor cells prior to the second reagent contacting with the tumor cells, or wherein the second reagent is contacted with the tumor cells prior to the first reagent contacting with the tumor cells.

43. The method of claim 41, wherein the first reagent is selected from a group consisting of: a myeloid derived suppressor cell recruitment inhibitor, a myeloid derived suppressor cell expansion inhibitor, a myeloid derived suppressor cell differentiation inhibitor, a myeloid derived suppressor cell activity inhibitor, a myeloid derived suppressor cell eliminator.

44. The method of claim 41, wherein the first reagent inhibits granulocytic myeloid derived suppressor cell.

45. The method of claim 41, wherein the first reagent is aldoxorubicin.

46. The method of claim 41, wherein the second reagent is selected from a group consisting of: an IL-8 antagonist, a CXCR1 inhibitor, and a CXCR2 inhibitor.

47. The method of claim 46, wherein at least one of the IL-8 antagonist, the CXCR1 inhibitor, and the CXCR2 inhibitor is selected from a group consisting of: an antibody, siRNA, miRNA, and a scFv fragment.

48. The method of claim 41, wherein the second reagent blocks at least two of the IL-8 signaling, the CXCR1 pathway, the CXCR2 pathway, and the myeloid derived suppressor cell.

49. The method of claim 41, further comprising determining expression of an EMT marker after contacting at least one of first and second reagents.

50. The method of claim 41, wherein the treatment is at least one of a chemotherapy, a radiation therapy, and an immune therapy.

51. The method of claim 41, further comprising contacting the tumor cell with a third reagent in a third amount that induces the tumor cell transformation from a mesenchymal to an epidermal state.

52. A method of treating a tumor cell in a patient, comprising:
preconditioning the tumor cell by contacting the tumor cell with a first reagent that suppresses a myeloid derived suppressor cell (MDSC) in a tumor microenvironment, and contacting the tumor cell with a second reagent that blocks at least one of IL-8 signaling, a CXCR1 pathway, a CXCR2 pathway;
wherein the first and second agents are administered in first and second amounts that prevent epidermal to mesenchymal transition of the tumor cell;
wherein the first reagent reduces or abrogates recruitment of MDSCs in the tumor and/or accumulation of MDSCs in the tumor, and/or inhibits expansion of MDSCs in the tumor, and
wherein the first reagent is distinct from the second reagent; and
treating the tumor cell with at least one of chemotherapy, radiation therapy, and an immune therapy.

53. The method of claim 52, wherein the first reagent is contacted with the tumor cells prior to the second reagent contacting with the tumor cells.

54. The method of claim 52, further comprising determining expression of an EMT marker after contacting at least one of first and second reagents.

55. The method of claim 52, wherein the first reagent is selected from a group consisting of: a myeloid derived suppressor cell recruitment inhibitor a myeloid derived suppressor cell expansion inhibitor, a myeloid derived suppressor cell differentiation inhibitor, a myeloid derived suppressor cell activity inhibitor.

56. The method of claim 52, wherein the first reagent is aldoxorubicin.

57. The method of claim 52, wherein the second reagent is selected from a group consisting of: an IL-8 antagonist, a CXCR1 inhibitor, and a CXCR2 inhibitor.

58. The method of claim 52, wherein the second reagent blocks at least two of the IL-8 signaling, the CXCR1 pathway, the CXCR2 pathway, and the myeloid derived suppressor cell.

59. The method of claim 52, wherein the chemotherapy is a metronomic low-dose chemotherapy, or wherein the immune therapy induces NK cell-mediated immune response and a T cell-mediated immune response.

60. The method of claim 52, wherein the immune therapy is a cell-based therapy that comprises modified NK cells, modified T cells, or an adenovirus is administered to the cells to produce neoepitopes displayed on the cells.
